(19) United States
(12) Patent Application Publication
(10) Pub. No.: US 2010/0284912 A1
(43) Pub. Date: Nov. 11, 2010

(54) HOMOGENIZATION OF A RADIOPHARMACEUTICAL USING SONIFICATION AND/OR ROTOR-STATOR TECHNOLOGY TO PRODUCE A HOMOGENOUS SUSPENSION, EMULSION, MIXTURE OR SOLID SUSPENSION OF IMMISCIBLE INGREDIENTS

(75) Inventors: Peter J. Oehlberg, Plainsboro, NJ (US); Ernst Schramm, Milltown, NJ (US)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: BRACCO DIAGNOSTICS INC., Princeton, NJ (US)

(21) Appl. No.: 12/745,505

(22) PCT Filed: Dec. 3, 2008
(86) PCT No.: PCT/US08/85361

§ 371 Date: Jul. 14, 2010

Related U.S. Application Data
(60) Provisional application No. 60/992,162, filed on Dec. 4, 2007.

(51) Int. Cl. A61K 51/12; A61P 5/00; A61P 35/00

(52) U.S. Cl. 424/1.61; 424/1.11

(57) ABSTRACT

A homogenous dispersion of aqueous droplets of a radiopharmaceutical or radiodiagnostic agent within a non-aqueous medium and method of making the same is provided. The homogenous dispersion is largely free of impurities and constitutes a substantially uniform distribution of sodium thiosulfate and radiopharmaceutical which is stable for a time sufficient to fill and solidify within gel capsules. In making the homogenous dispersion, dry sodium thiosulfate and dry potassium phosphate dibasic anhydrous are uniformly dispersed within non-aqueous, liquid polyethylene glycol.

### CROSS-REFERENCE TO RELATED APPLICATIONS

This application claims priority to and benefit of U.S. provisional application No. 60/992,162 filed Dec. 4, 2007 which is incorporated herein by reference.

### FIELD OF THE INVENTION

The present invention relates to compositions of and methods of making substantially homogenous stable dispersions of radiopharmaceuticals in non aqueous mediums such as PEG.

### BACKGROUND

Radiopharmaceuticals are useful in both diagnostic and therapeutic use. For example, Sodium Iodide (I-131) is useful in antihyperthyroid therapy and antineoplastic therapy. Whereas, Technetium (Tc-99m) Mebrofenin is useful in diagnostics, particularly as a hepatobiliary imaging agent used in cholescintigraphy. Similarly, Technetium Tc-99m Medronate and Technetium Tc-99m Pyrophosphate are useful as bone imaging agents. Cyanocobalamin Co-57 is used for the diagnosis of pernicious anemia and as a diagnostic adjunct in other defects of intestinal vitamin B12 adsorption.

Radiopharmaceuticals for oral administration are often formulated in the form of gelatin capsules, also referred to as gel caps. Such gel caps can be made by filling the capsule cavity with a mixture of water insoluble liquefied polyethylene glycol (PEG) in which aqueous spheres or droplets of radiopharmaceutical and attendant excipients are dispersed. Such dispersions are nonhomogenous. The mixture within the gel cap is then allowed to cool, solidifying the PEG around the aqueous droplets.

A typical procedure involves liquefying PEG by heating, slowly adding powered Sodium Thiosulfate and then powdered Potassium Phosphate Dibasic Anhydrous while stirring the liquefied PEG. A pH check can be performed prior to the addition of aqueous radiopharmaceutical. Buffered, or in other prior methods non-buffered, aqueous radiopharmaceutical is then added while stirring. The gel caps are then filled with the resulting mixture.

The gel caps are then assayed to determine their potency (i.e. mCi of radioactivity) and sorted according to the level of radioactivity present. Radioactivity in individual capsules filled according to these methods can vary from as much as 200 mCi to as little as 2 mCi per capsule for therapeutic capsules and from 5 μCi to 100 μCi for diagnostic capsules, even though they are filled with the same volume from the same batch. Thus, gel caps must be individually assayed and sorted according to activity. Some methods omit the assay step, but do nothing to reduce the variability between individual capsules. Furthermore, under such methods, a percentage of gel caps may be deformed and/or leak during storage due to the presence of larger aqueous droplets near the inner surface of the gel cap which would dissolve the gelatin wall of the capsule.

Furthermore, during the mixing procedures employed in existing methods, salt clumps form along the bottom and sides of the mixing vessel. It is believed this is due to the difficulty of dispersing aqueous salts within a liquid non-aqueous environment, e.g. liquid polyethylene glycol.

Thus a composition of and method of forming a homogenous suspension, emulsion, mixture or solid suspension of radiopharmaceutical which provides a uniform amount of radiopharmaceutical per gel cap and which reduced or eliminated the frequency of deformed and/or leaking gel caps was desired.

### SUMMARY OF THE INVENTION

The foregoing provides a non-exclusive list of the objectives achieved by the present invention:

It is a primary object of the invention to provide a method of formulating a substantially homogenous dispersion of radiopharmaceutical and excipients within a medium which is immiscible with the dispersed radiopharmaceutical and excipients.

It is a further object of the invention to provide a method of formulating a substantially homogenous dispersion of substantially aqueous radiopharmaceutical and excipients within a substantially non-aqueous medium.

It is a further object of the invention to provide a method of formulating a substantially homogenous dispersion of substantially aqueous radiopharmaceutical and excipients within a substantially non-aqueous medium by sonifying or homogenizing a mixture of the aqueous or substantially aqueous radiopharmaceutical and excipients or emulsified radiopharmaceutical and excipients with a substantially non-aqueous medium.

It is also a primary object of the invention to provide a composition comprising a substantially homogenous dispersion of radiopharmaceutical and excipients within a medium which is immiscible with the dispersed radiopharmaceutical and excipients.

It is a further object of the invention to provide a composition comprising a substantially homogenous dispersion of aqueous or substantially aqueous radiopharmaceutical and excipients or emulsified radiopharmaceutical and excipients with a substantially non-aqueous medium.

It is a further object of the invention to provide a composition comprising a substantially homogenous dispersion of aqueous or substantially aqueous radiopharmaceutical and excipients or emulsified radiopharmaceutical and excipients with a substantially non-aqueous medium wherein the radiopharmaceutical and excipients is in the form of dispersed spheres or fine droplets which retain their dispersion for an amount of time sufficient to enable the filling of multiple gel capsules with substantially uniform amounts of radiopharmaceutical and excipients.

These and other objects, features and advantages may be achieved by sonifying or homogenizing a mixture of an aqueous solution of radiopharmaceutical which has been mixed with liquefied PEG and any other necessary excipients.

These and other objects, features and advantages are also achieved by sonifying or homogenizing a mixture of an aqueous solution of radiopharmaceutical which has been mixed with liquefied PEG and any other necessary excipients where the mixture has an immiscible bottom layer and a top layer of substantially unmixed PEG.

### BRIEF DESCRIPTION OF THE DRAWINGS

FIG. 1A is a flow chart of a method of preparing gel capsules containing a homogenous dispersion of radiopharmaceutical fixed within PEG according to an exemplary embodiment of the present invention.

FIG. 1B is a flow chart of a method of preparing gel capsules containing a homogenous dispersion of radiopharmaceutical fixed within PEG according to another exemplary embodiment of the present invention.

FIG. 2 is a photograph of a dispersion of aqueous radiopharmaceutical and excipients dispersed within liquid PEG according to an exemplary embodiment of the present invention.

FIG. 3 is a drawing (not to scale) of a dispersion of aqueous radiopharmaceutical and excipients dispersed within liquid PEG according to an exemplary embodiment of the present invention.

FIG. 4A is a paper chromatography radioactivity profile of a capsule containing a dispersion of aqueous radiopharmaceutical and excipients dispersed within liquid PEG at 0 hours according to an exemplary embodiment of the present invention.

FIG. 4B is a paper chromatography radioactivity profile of a capsule containing a dispersion of aqueous radiopharmaceutical and excipients dispersed within liquid PEG at 24 hours according to an exemplary embodiment of the present invention.

FIG. 4C is a paper chromatography radioactivity profile of a capsule containing a dispersion of aqueous radiopharmaceutical and excipients dispersed within liquid PEG at 72 hours according to an exemplary embodiment of the present invention.

FIG. 5A is a Fourier transform infrared spectroscopy (“FT-IR spectra”) of a dispersion according to an exemplary embodiment of the present invention.

FIG. 5B is a FT-IR spectra of a mixture of PEG 3350, potassium phosphate dibasic and sodium thiosulfate pentahydrate in a ratio of 8.52:1.06:0.71, according to an exemplary embodiment of the present invention.

FIG. 5C is a FT-IR spectra of 3350 PEG.

FIG. 6A is a Differential Scanning Calorimetry thermogram (“DSC thermogram”) of a dispersion according to an exemplary embodiment of the present invention.

FIG. 6B is a DSC thermogram of a mixture of PEG 3350, potassium phosphate dibasic and sodium thiosulfate pentahydrate in a ratio of 8.52:1.06:0.71, according to an exemplary embodiment of the present invention.

FIG. 6C is a DSC thermogram of 3350 PEG.

FIG. 7A is a Thermogravimetric Analysis (“TGA thermogram”) of a dispersion according to an exemplary embodiment of the present invention.

FIG. 7B is a TGA thermogram of a mixture of PEG 3350, potassium phosphate dibasic and sodium thiosulfate pentahydrate in a ratio of 8.52:1.06:0.71, according to an exemplary embodiment of the present invention.

FIG. 7C is a TGA thermogram of 3350 PEG.

### DETAILED DESCRIPTION

The present invention is directed, inter alia, to the formation of substantially homogenous dispersions of radiopharmaceuticals within PEG. For example, the radiopharmaceutical may comprise —I¹³¹, or any other radioactive ion used for diagnostic use, in vitro labeling or therapeutic use. The radiopharmaceutical may also comprise a radiolabeled compound used for diagnostic use, in vitro labeling or therapeutic use.

Substantially homogenous dispersions of radiopharmaceuticals within PEG in accordance with embodiments of the invention are made by first layering powdered PEG onto the bottom of a suitable vessel. Any suitable vessel may be used; however, in a preferred embodiment, the vessel is cylindrical. The first layer of PEG is then covered by a second layer of powdered PEG, powdered Sodium Thiosulfate and powdered Potassium Phosphate Dibasic Anhydrous added in series while stirring the second layer until the second layer is substantially mixed; a third layer of powdered PEG is then added on top of the second layer. The PEG, Sodium Thiosulfate and Potassium Phosphate Dibasic Anhydrous are preferably in the form of fine powders. In a preferred embodiment, the fine powder includes controlled size particles of Sodium Thiosulfate or Potassium Phosphate Dibasic, preferably particles of less than 590 μm.

Powdered Sodium Thiosulfate suitable for use in the present invention can be formed, for example, by pulverizing Sodium Thiosulfate using a mortar and pestle followed by passing the resulting material through a screen, preferably mesh 32 screen, to generate particles of Sodium Thiosulfate which are about 590 μm or less in size. Similarly, Powdered Potassium Phosphate Dibasic suitable for use in the present invention can be formed, for example, by pulverizing Potassium Phosphate Dibasic using a mortar and pestle followed by passing the resulting material through a screen, preferably mesh 32 screen, to generate particles of Potassium Phosphate Dibasic which are about 590 μm or less in size.

Suitable PEGs useful in embodiments of the present invention include PEG 3350, PEG 4000 and those PEGs with melting temperatures above room temperature and below temperatures which would alter or harm the radiopharmaceutical. Preferably the PEG melting temperature would be above the environmental temperatures to which the gel capsules will be exposed. Persons of ordinary skill in the art would be able to select suitable PEGs by consulting reference manuals detailing their melting temperatures and characteristics. PEG 3350, which is in powder form, is particularly preferred.

The aforementioned three layers are then heated until they melt to form a suspension of powdered Sodium Thiosulfate and powdered Potassium Phosphate Dibasic Anhydrous within the liquid PEG. Heat is supplied as necessary throughout the mixing process such that the PEG is kept in a liquid state. Upon melting, the liquid PEG, powdered Sodium Thiosulfate and powdered Potassium Phosphate Dibasic Anhydrous are stirred, preferably by magnetic stir bar, to achieve a substantially uniform distribution of Sodium Thiosulfate and powdered Potassium Phosphate Dibasic Anhydrous within the liquid PEG. Stirring is preferably maintained throughout the foregoing steps.

In one embodiment the pH of the mixture is assayed. In a preferred embodiment, particularly where the radiopharmaceutical comprises radioactive iodine, a target pH is set. In a particularly preferred embodiment, the pH is greater than 6.8. If this pH target is not met, the pH may be adjusted or the batch may be discarded and the aforementioned procedure repeated until a batch with pH at the target level (e.g. greater than 8.6) is obtained.

After the powdered Sodium Thiosulfate and powdered Potassium Phosphate Dibasic Anhydrous are suspended in PEG and the pH has been verified as necessary, radiopharmaceutical, dissolved in an aqueous liquid medium, preferably dissolved in a 0.05 N NaOH buffer, is added to the mixture of, substantially suspended Sodium Thiosulfate, substantially suspended Potassium Phosphate Dibasic Anhydrous, optional aqueous base and liquefied PEG. Volume may be adjusted with 0.05 N NaOH such that the total volume is consistent from batch to batch.

While the mixture is stirring it is subjected to one or more of sonification with a sonifier and homogenization with a homogenizer until a dispersion of substantially uniform, fine aqueous spheres or droplets are formed. The dispersion of aqueous spheres or droplets preferably remains stable for between 20 and 30 minutes (or at least as long as is necessary to fill and solidify within gel capsules). The mixture is then withdrawn from the mixing vessel and aliquoted into gel capsules which are allowed to cool, suspending the dispersion of aqueous spheres or droplets within solid PEG.

In one preferred embodiment, sonification is carried out for 3-7 minutes at 13 Watts. A variety of sonifiers may be used in accordance with the present invention, including, but not limited to a commercial sonifier such as the Hielscher™ UP 100H sonifier (Hielscher USA, Inc, Ringwood, N.J.).

In other embodiments, rotor-stator type homogenization may be used in place of, in conjunction with, or preceding or following sonification. Homogenization may be carried out for 3-20 minutes. A variety of homogenizers may be used in accordance with the present invention, including, but not limited to commercial homogenizers such as the OMNI TH-01 (OMNI International, Inc, Marietta, Ga.)

In one embodiment of the present invention, the use of a third layer of powdered PEG placed on top of the second layer of powdered PEG, powdered Sodium Thiosulfate and powdered Potassium Phosphate Dibasic Anhydrous (or other water soluble powdered salts or excipients), substantially allows the powdered Sodium Thiosulfate and powdered Potassium Phosphate Dibasic Anhydrous, or other water-soluble powders, to be washed down the sides of the mixing vessel as the PEG is melted. Preferably the third layer of powdered PEG is applied such that the PEG powder coats the sides of the vessel as well as covering the second layer, thus ensuring that as it melts, the third layer of PEG washes the vessel walls clear of powdered salts (Sodium Thiosulfate and Potassium Phosphate Dibasic Anhydrous). This washing allows substantially all of the water soluble compounds to be contained within the liquid PEG, preventing them from forming clumps along the sides of the vessel.

In another embodiment of the present invention, the first layer of powdered PEG substantially prevents powdered Sodium Thiosulfate and powdered Potassium Phosphate Dibasic Anhydrous, or other water-soluble powders, from collecting at the bottom edge of the mixing vessel, preventing the water soluble compounds from forming clumps along the bottom edge of the vessel.

Preventing clump formation allows for greater contact between the added aqueous phase consisting of the radiopharmaceutical (eg. I-131) in an aqueous liquid media such as 0.05N Sodium Hydroxide and the water-soluble powders dispersed within the liquid PEG, thus facilitating dissolution into the aqueous phase.

Importantly, the type of salts used may vary depending upon the formulation and/or the radiopharmaceutical being formulated. The methods of the present invention are useful with a variety of radiopharmaceutical formulations and with a variety of water soluble salts and excipients. In one preferred embodiment discussed above, powdered Sodium Thiosulfate and powdered Potassium Phosphate Dibasic Anhydrous are used however, other suitable salts may be added or substituted for these. Additionally, the methods of the present invention can be used with radiopharmaceutical formulations containing emulsifiers such as surfactants. Surfactants are especially useful where the radiopharmaceutical has somewhat limited or no solubility in water.

Similarly, the present invention is useful in the filling of any capsule which is filled with a liquid and is thus not intended, nor limited to gel capsules per se.

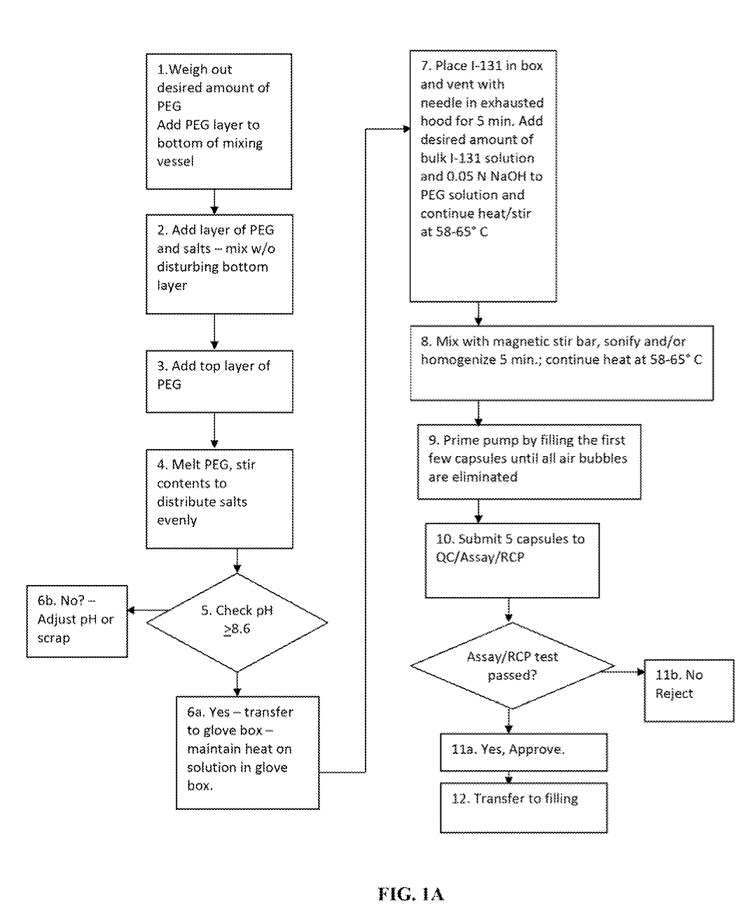

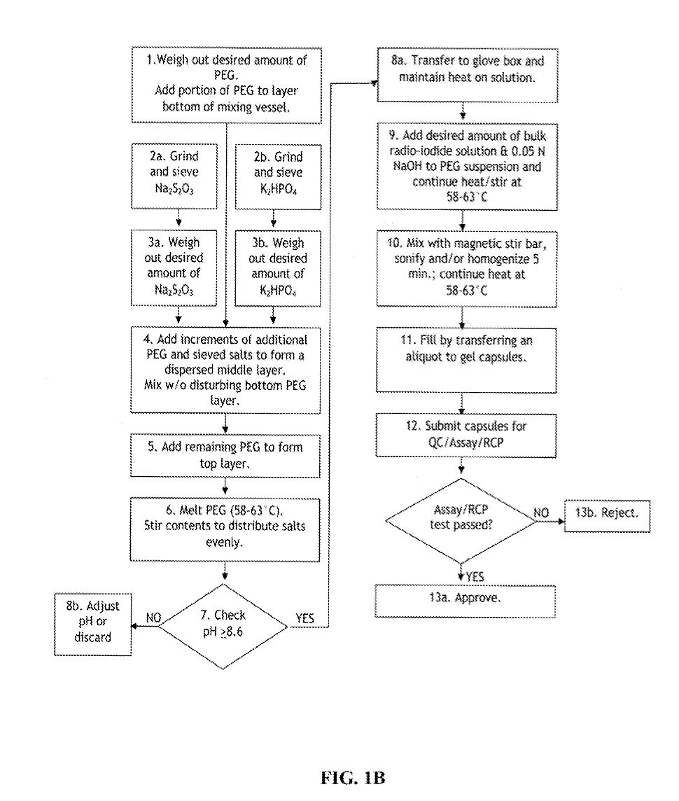

Reference is now made to FIGS. 1A and B which provide flow charts describing the steps of formulating radiopharmaceutical gel capsules in accordance with certain embodiments of the present invention:

Referring now to FIG. 1A, at step 1 the PEG is weighed out according to the formulation being used and a bottom layer of PEG is added to the mixing vessel. At step 2, a layer, or multiple layers of PEG and powdered salts are added to the mixing vessel and mixed without substantially disturbing all of the bottom layer. At step 3, a top layer of PEG is added to the mixing vessel. Step 4 involves using heat (58-65° C.) to melt the PEG and stirring contents to substantially evenly distribute the salts; stirring is continuous, but may be temporarily interrupted for process interventions, until the batch is aliquoted into individual gel capsules. A pH check is performed at step 5. If the pH exceeds 8.6, the procedure is continued (step 6a). If the pH is 8.6 or less, the batch is discarded and steps 1-6 are repeated (step 6b). At step 6a, the mixing vessel is transferred to a glove box (to prevent exposure of persons and the environment to radioactivity) where mixing and heating are continued. At step 7 the desired amount of bulk radiopharmaceutical (e.g. I-131) solution is added, depending on the desired radioactivity level per capsule, and an aqueous liquid medium such as 0.05 N NaOH may be added to the PEG solution to finalize the volume. Heating and stirring at 58-65° C. is continued. Step 8 involves continued heating at 58-65° C. and stirring with the magnetic stir bar, as well as sonification and/or rotor-stator homogenization for a period of time, preferably a period of time, preferably from 3-20 minutes to achieve a stable homogenized dispersion of fine aqueous spheres or spherically shaped droplets. Steps 9-12b involve filling, cooling and testing of the finished gel capsules.

In another embodiment, the need to stir the contents is eliminated or substantially reduced by using a rotor-stator homogenizer. In a further embodiment, if the pH is outside the target level (e.g. below 8.6), it may be adjusted (to >8.6), by, for example, using a basic solution instead of discarding a batch which has pH <8.6. In yet another embodiment, pH is not checked.

Referring now to FIG. 1B, at step 1 the PEG is weighed out according to the formulation being used and a bottom layer of PEG is added to the mixing vessel. At steps 2a and 2b the particle size of the slats is controlled as the Sodium Thiosulfate and Potassium Phosphate Dibasic are respectively ground and sieved to a powder. At steps 3a and 3b, the amounts of Sodium Thiosulfate and Potassium Phosphate dibasic are weighed out according to the formulation being used. Then, at step 4, a middle layer of PEG and salts are added to the mixing vessel and mixed without substantially disturbing all of the bottom layer. Alternatively, the salts and PEG can be mixed separately and added to the vessel. At step 5, a top layer of PEG is added to the mixing vessel. Step 6 involves using heat (58-65° C.) to melt the PEG and stirring contents to substantially evenly distribute the salts; stirring is continuous, but may be temporarily interrupted for process interventions, until the batch is aliquoted into individual gel capsules. A pH check is performed at step 7. If the pH exceeds 8.6, the procedure is continued (step 8a). If the pH is 8.6 or less, the pH is adjusted in step 8b or the batch is discarded and steps 1-7 are repeated to make another batch. At step 8a, the mixing vessel is transferred to a glove box (to prevent exposure of persons and the environment to radioactivity) where mixing and heating are continued. At step 9 the desired amount of bulk radiopharmaceutical (e.g. I-131) solution is added, depending on the desired radioactivity level per capsule, and an aqueous liquid medium such as 0.05 N NaOH may be added to the PEG solution to finalize the volume. Heating and stirring at 58-65° C. is continued. Step 10 involves continued heating at 58-65° C. and stirring with the magnetic stir bar, as well as sonification and/or rotor-stator homogenization for a period of time, preferably a period of time, preferably from 3-20 minutes to achieve a stable homogenized dispersion of fine aqueous spheres or spherically shaped droplets. Steps 11-13b involve filling, cooling and testing of the finished gel capsules.

In another embodiment, the need to stir the contents is eliminated or substantially reduced by using a rotor-stator homogenizer. In a further embodiment, if the pH is outside the target level (e.g. below 8.6), it may be adjusted (to >8.6), by, for example, using a basic solution instead of discarding a batch which has pH <8.6. In yet another embodiment, pH is not checked.

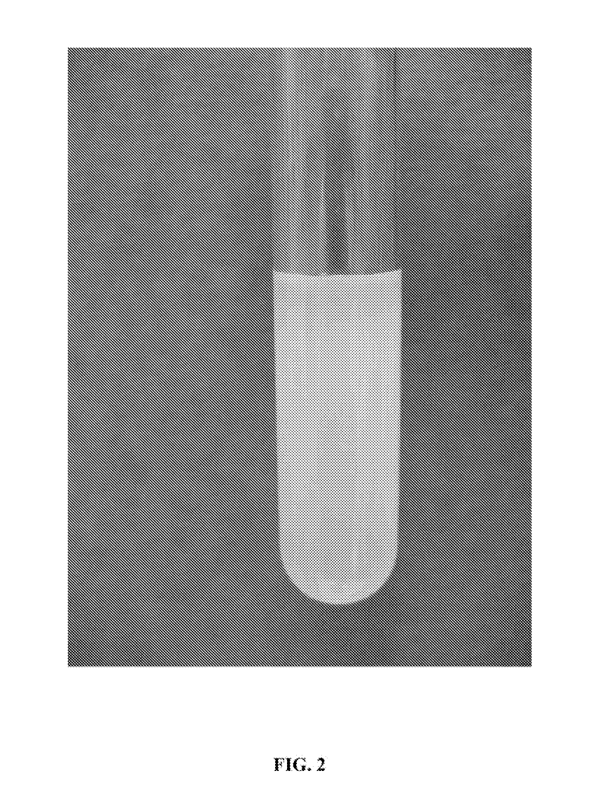

Referring now to FIG. 2, a photograph of a dispersion of fine aqueous spherically shaped droplets made in accordance with the present invention is presented. As shown in FIG. 2, the dispersion appears as a very fine homogenous haze of aqueous droplets within the PEG medium.

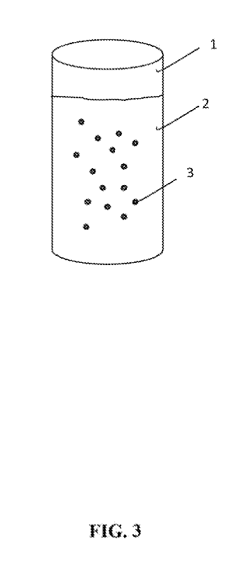

Referring now to FIG. 3, a vial, 1 containing a dispersion of fine aqueous spherically shaped droplets, 3, in liquid PEG, 2, made in accordance with the present invention is depicted showing the substantially uniform distribution of fine aqueous spherically shaped droplets. Droplets, 3, are not shown to scale. The uniform distribution of aqueous droplets result in a homogeneous dispersion of both the salts (e.g. Sodium Thiosulfate) and the radiopharmaceutical (e.g. ¹³¹I).

The features and advantages of certain embodiments of the present invention will be further apparent from the following examples.

### Example 1

Gel capsules were filled with a homogenous dispersion of aqueous solution in PEG prepared using a sonifier. The homogenous dispersion was formulated in a 10 mL batch as follows:

| Components | Amount | |
|---|---|---|
| PEG 3350 (powdered) | 8.52 | g |
| Sodium Thiosulfate (pulverized) | 0.71 | g |
| Potassium Phosphate Dibasic Anhydrous | 1.06 | g |
| (pulverized) | | |
| 0.05N NaOH | ≦1.31 | mL |
| 0.05N NaOH | to 10 | mL |

The homogenous dispersion was prepared by creating a suspension of the water soluble components in aqueous solution in a medium of molten PEG. The formulation was carried out in a 20 mL round serum tubing vial. A first layer of about ¼ to ⅓ of the powdered PEG 3350 was placed onto the bottom of the vial. A second layer of about ½ to ⅓ of the PEG 3350, the pulverized Sodium Thiosulfate and the pulverized Potassium Phosphate Dibasic Anhydrous was added to the vial and manually stirred to mix without substantially disturbing the first layer. A third layer of the remaining PEG 3350 was added to the vial on top of the second layer.

The vial was then sealed with a septum-like closure through which components could be delivered by piercing the stopper with a syringe, glass tube or other suitable device. A heating mantle bath was placed in contact with the vial and the vial was placed onto a magnetic stir plate. The contents of the vial were then heated to 58-65° C. to liquefy the PEG 3350. A magnetic stir bar was placed in the vessel. Upon liquefying of the PEG 3350, the magnetic stirrer was activated and the mixture was stirred vigorously for 20 minutes to distribute the pulverized Sodium Thiosulfate and the pulverized Potassium Phosphate Dibasic Anhydrous within the liquid PEG.

1.31 mL 0.05N Sodium Hydroxide (as a placebo for the radiopharmaceutical) was added to the vial while continuing to stir and maintain heat at 58-62° C. A make-up volume of 0.05 N NaOH was added until the total volume was 10 mL.

A Hielscher™ UP 100H (Hielscher USA, Inc, Ringwood, N.J.) sonifier was then introduced into the vial. The mixture was sonified at one cycle and an amplitude of 13 Watts for 3 minutes. Fine aqueous, homogenously dispersed spherically shaped droplets were then observed within the liquefied PEG.

A series of gel capsules were then filled while maintaining mixing with the magnetic stir bar.

Varying fill volumes ranged from 10 μL to 350 μL as determined by the total activity required per capsule. Gel capsules were then allowed to cool, solidifying the PEG.

### Example 2

Gel capsules were filled with a homogenous dispersion prepared using a rotor-stator homogenizer. The homogenous dispersion was formulated in a 10 mL batch as follows:

| Components | Amount | |
|---|---|---|
| PEG 3350 (powdered) | 8.52 | g |
| Sodium Thiosulfate (pulverized) | 0.71 | g |
| Potassium Phosphate Dibasic Anhydrous | 1.06 | g |
| (pulverized) | | |
| 0.05N NaOH | ≦1.31 | mL |
| 0.05N NaOH | to 10 | mL |

The homogenous dispersion was prepared by creating a suspension of the water soluble components in aqueous solution in a medium of molten PEG. The formulation was carried out in a nominal 10 mL processing vessel (a square screw-cap vial). A first layer of about ¼ to ⅓ of the powdered PEG 3350 was placed onto the bottom of the vial. A second layer of about ½ to ⅓ of the PEG 3350, the pulverized Sodium Thiosulfate and the pulverized Potassium Phosphate Dibasic Anhydrous was added to the vial and manually stirred to mix without substantially disturbing the first layer. A third layer of the remaining PEG 3350 was added to the vial on top of the second layer. A magnetic stir bar was placed in the vessel. A heating mantle bath was placed in contact with the vial and the vial was placed onto a magnetic stir plate. The contents of the vial were then heated to 58-65° C. to liquefy the PEG 3350. Upon liquefying of the PEG 3350, the magnetic stirrer was activated and the mixture was stirred vigorously for 20 minutes to distribute the pulverized Sodium Thiosulfate and the pulverized Potassium Phosphate Dibasic Anhydrous within the liquid PEG.

The vial was then sealed with a screw-cap with center opening through which components could be delivered. 1.31 mL of 0.05 N NaOH (as a placebo for the radiopharmaceutical) was added to the vial while continuing to stir and maintain heat at 58-62° C. OMNI TH-01 (OMNI International, Inc, Marietta, Ga.) rotor-stator homogenizer plastic disposable probe was then introduced into the vial through the center opening of the screw-cap. The mixture was homogenized for 7 minutes at 35000 RPM speed. Fine aqueous, homogenously dispersed spherically shaped droplets were then observed within the liquefied PEG.

A series of gel capsules were then filled while maintaining mixing with the magnetic stir bar. Varying fill volumes ranged from 10 μL to 350 μL, as determined by the total activity required per capsule. Gel capsules were then allowed to cool, solidifying the PEG.

### Example 3

### Prophetic

The following components are used with the same equipment, under the same conditions, as mentioned in Example 1. I-131 will be substituted for placebo as follows:

| Component | Amount |
|---|---|
| Sodium RadioIodide I-131 | x mL ≦ 1.31 mL |
| (2200-4400 mCi) (in 0.05N NaOH) | |

Fine aqueous, homogenously dispersed spherically shaped droplets will be observed within the liquefied PEG.

### Example 4

### Prophetic

The following components are used with the same equipment, under the same conditions, as mentioned in Example 2. I-131 will be substituted for placebo as follows:

| Component | Amount |
|---|---|
| Sodium RadioIodide I-131 | x mL ≦ 1.31 mL |
| (2200-4400 mCi) (in 0.05N NaOH) | |

Fine aqueous, homogenously dispersed spherically shaped droplets will be observed within the liquefied PEG.

### Example 5

A first batch (Batch A) of nine gelatin capsules were prepared according Example 2, each containing 80 uL of a radiopharmaceutical formulation containing ¹²⁵I. The radioactivity of each of the Batch A capsules was measured. A second batch (Batch B) of three additional capsules were prepared according to the same procedure, but filled after a 1.5 hour holding period. The radiopharmaceutical formulation was kept at 63° C. without stirring during the holding period. The radioactivity of each of the Batch B capsules was then measured. Table 1 depicts the radioactivity of the capsules from Batch A and Table 2 depicts the radioactivity of the capsules from Batch B, measured in μCi.

TABLE 1

| Capsule Number | Radioactivity in μCi(¹²⁵I) |
|---|---|
| 1 | 13.27 |
| 2 | 12.01 |
| 3 | 13.20 |
| 4 | 12.10 |
| 5 | 13.17 |
| 6 | 13.40 |
| 7 | 13.28 |
| 8 | 12.45 |
| 9 | 13.24 |
| Average | 12.90 |
| STDEV | 0.55 |
| CV % | 4.3 |

TABLE 2

| Capsule Number | Radioactivity in μCi(¹²⁵I) |
|---|---|
| 1 | 12.93 |
| 2 | 12.21 |
| 3 | 12.83 |
| Average | 12.66 |
| STDEV | 0.39 |
| CV % | 3.08 |

As shown in Table 1 the average radioactivity of the nine prepared capsules was 12.9 μCi with a Coefficient of Variation (CV) of 4.3%. Referring now to Table 2, the average radioactivity of the three capsules prepared after a 1.5 hour holding time was 12.66 μCi with a CV 3.1%, which is within 2% of the average for the nine capsules which were filled at hour 0.

### Example 6

The Radiochemical Purity (RCP) of the Batch A capsules of Example 5 was determined by paper chromatography at times 0, 24 and 72 hours.

TABLE 3

| Capsule Holding | Radiochemical | Impurity at | Impurity at |
|---|---|---|---|
| Time (hours) | Purity % | Origin % | R_{f} 0.7% |
| 0 | 99.04 | 0.01 | 0.00 |
| 24 | 98.06 | 0.06 | 1.19 |
| 48 | 97.74 | 0.09 | 1.44 |
| 72 | 98.05 | 0.05 | 1.90 |

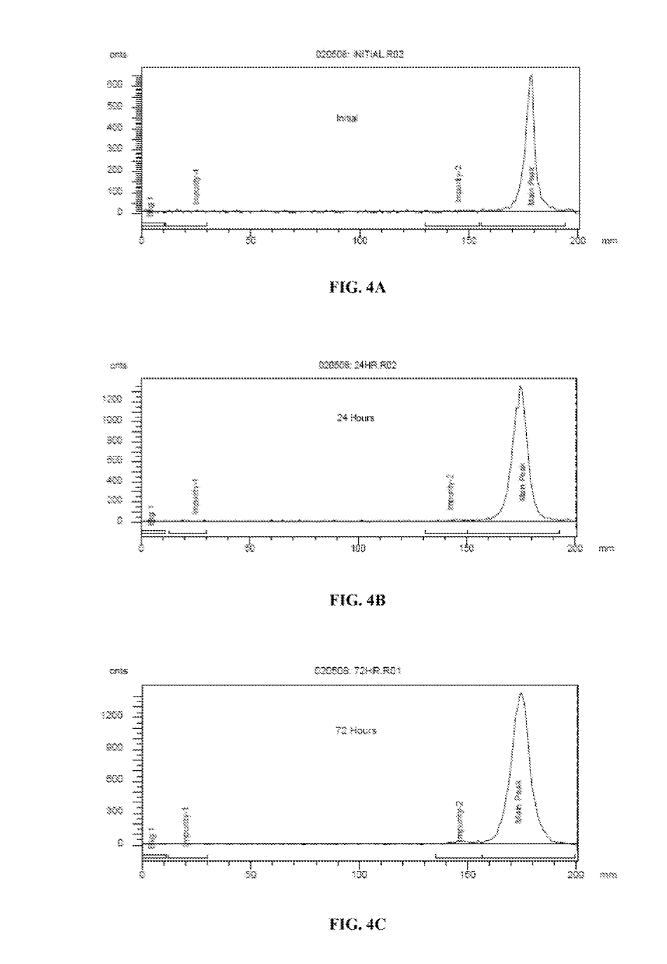

As shown in Table 3, the capsules remained stable with RCPs >98% during capsule holding times of 0, 24 and 72 hours. As also shown in Table 3 and FIGS. 4A-C, impurities were negligible, with impurities at the origin remaining below 0.1% at all timepoints and impurities at the solvent front remaining below 2% at all time points.

### Example 7

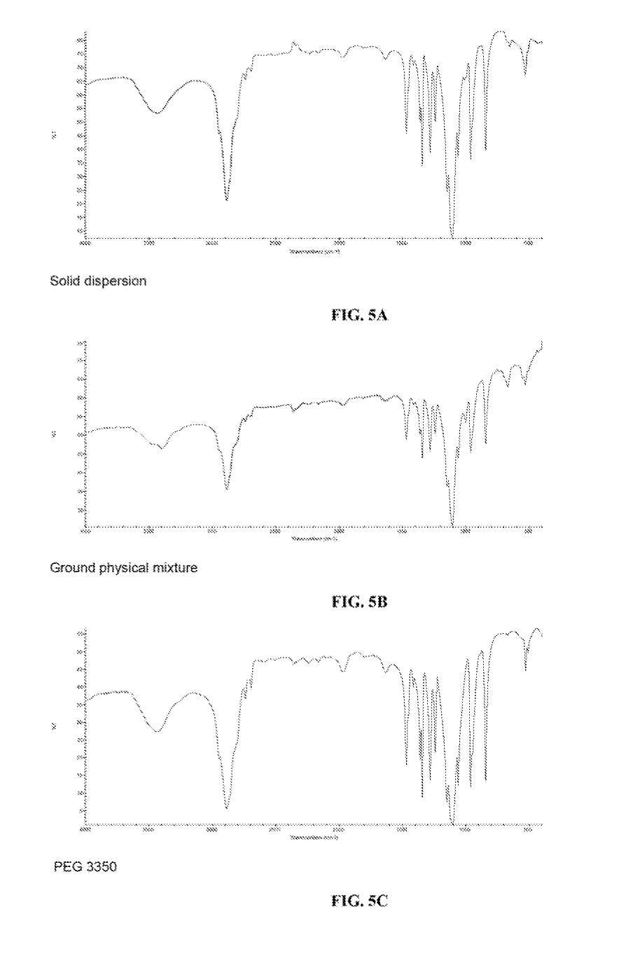

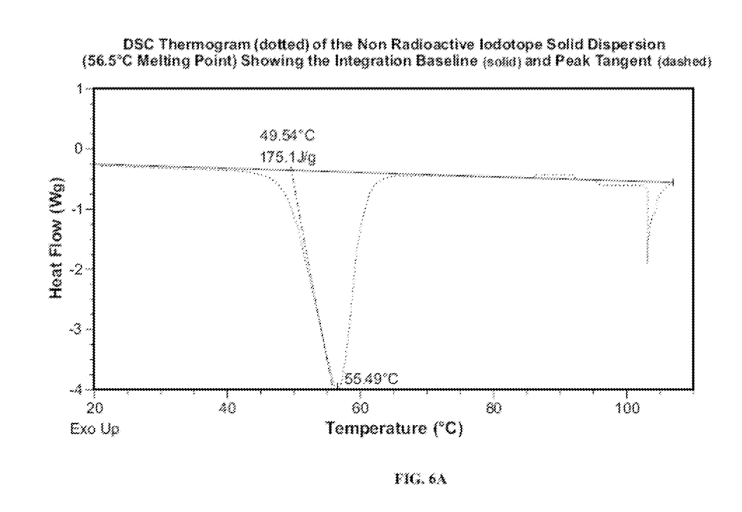

As shown in FIGS. 5A-C, FT-IR Spectra were collected for three samples: a solid dispersion, ground physical mixture and PEG 3350, with 1 mg of sample per 300 mg KBr pellet. The solid dispersion was the product resulting from the method shown in Example 2. The ground physical mixture was a mix of PEG 3350, potassium phosphate dibasic and sodium thiosulfate pentahydrate in a ratio of 8.52:1.06:0.71.

The spectra for all of the above samples were qualitatively identical, with the spectra for the solid dispersion and the ground physical mixture each showing minor bands corresponding to sodium thiosulfate and potassium phosphate dibasic which were also present in the control spectra for sodium thiosulfate and potassium phosphate dibasic. Thus the process of the present invention yields a suitably pure product with minor insignificant impurities.

### Example 8

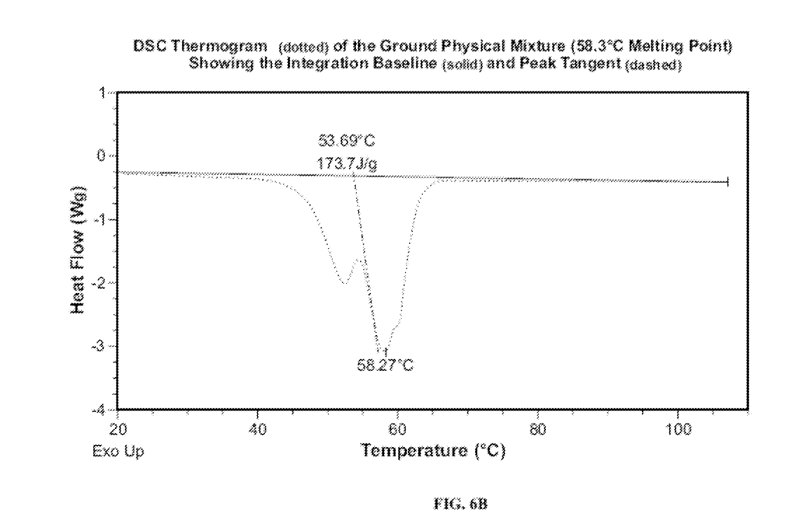

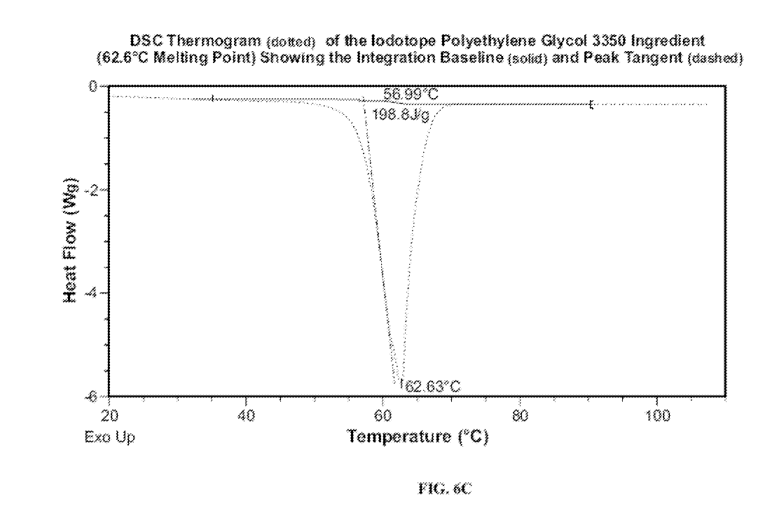

Referring now to FIGS. 6A-C, DSC thermograms were collected for the three samples of Example 7: a solid dispersion, ground physical mixture and the PEG 3350. 10 mg was collected for each sample. A heating ramp of 10° C./min from room temperature to 120° C. was used in the analysis. Melting points were determined for each of the samples, with PEG 3350 melting at 63° C., and the solid dispersion and ground physical mixture melting at 57-58° C. These findings show that the formulations of the present invention melt at temperatures suitable for manufacturing.

### Example 9

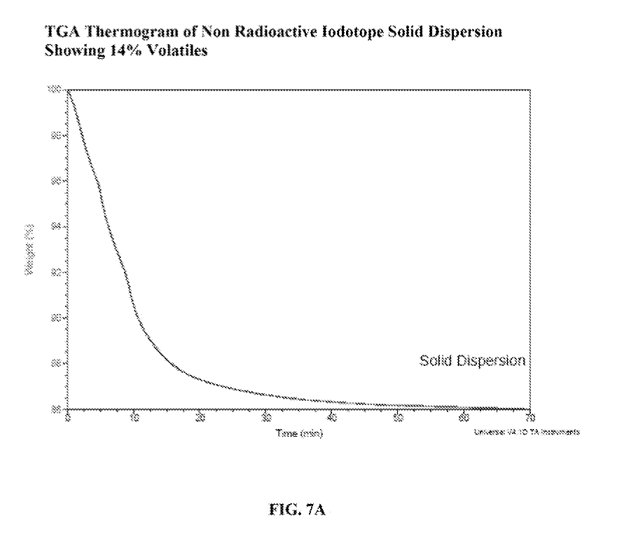

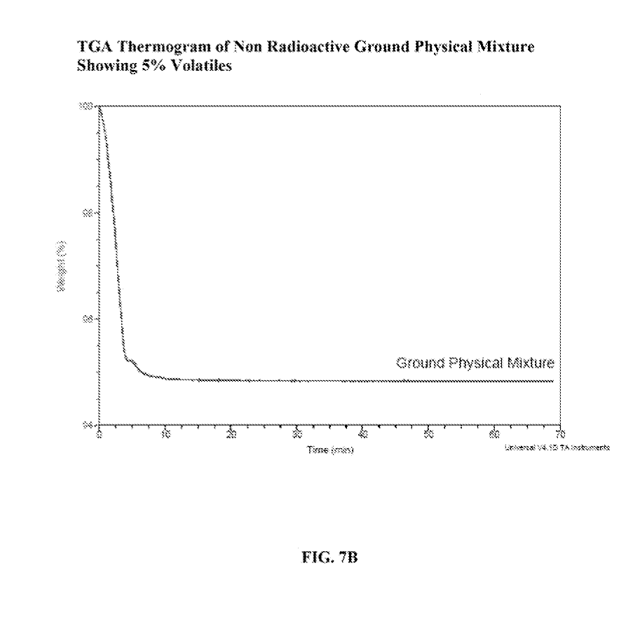

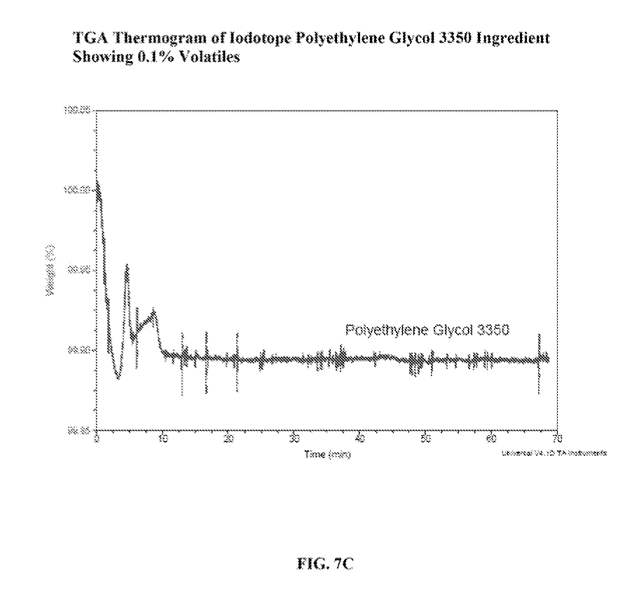

Thermo Gravimetric Analyzer (TGA) thermograms were collected for the three samples of Example 7: a solid dispersion, ground physical mixture and the PEG 3350 and for samples of Sodium Thiosulfate and Potassium Phosphate Dibasic, as shown in FIGS. 7A-C. 10 mg was collected for each sample. A heating ramp of 10° C./min from room temperature to 110° C. with an isothermal hold at 110° C. for 60 min was used in the analysis. Table 4 below depicts the % volatiles (water) which were found versus the theoretical values.

TABLE 4

| | % Volatiles | % Volatiles |
|---|---|---|
| Sample | Found | Theoretical |
| Solid Dispersion (improved | 14 | 13 |
| formulation process) | | |
| Ground Physical Mixture (no | 5 | 4 |
| water added) | | |
| PEG 3350 | 0.1 | <1 |
| Sodium Thiosulfate | 33 | 32-37 |
| Potassium Phosphate Dibasic | 2.7 | 1 |

Since percent volatiles were essentially almost equal to theoretical calculations, the method of the present invention is shown to produce a radiopharmaceutical or radiodiagnostic with stable component concentrations.

### Example 10

A 5 ml capacity pycnometer was used to determine the density and specific gravity of a non-radioactive formulation prepared in accordance with Example 2 at 60° C. The density of the formulation was found to be 1.166 g/cm³ and the specific gravity was found to be 1.186. Thus, the formulation, having a density and specific gravity just above that of water, is capable of being filled into gel capsules using standard manufacturing techniques.

### Example 11

A distribution study was performed on a non radioactive formulation prepared in accordance with Example 2 (Batch B) which was subjected to a hold period of 1.5 hours prior to the filling of gel capsules, to determine the proportion of sodium thiosulfate in the aqueous and PEG layers. As shown in Table 5 below, sodium thiosulfate was uniformly distributed throughout the formulation. In fact, virtually 100% of the sodium thiosulfate was recovered in the uniform homogenized preparation.

TABLE 5

| | % Thiosulfate Recovery | | |
|---|---|---|---|
| | Control Spiked | Formulation | Spiked |
| Sample | Water Layer | Suspension | Formulation |
| 1 | 97.07 | 106.37 | 97.85 |
| 2 | — | 110.04 | 97.63 |
| 3 | — | 106.37 | 97.51 |
| Average | 97.1 | 107.6 | 97.7 |
| STDEV | | 2.1 | 0.2 |
| CV (%) | | 2.0 | 0.2 |

The uniform distribution of sodium thiosulfate throughout the formulation allows the iodide to be protected by the thiosulfate by maximizing the amount of thiosulfate in contact with iodide.

The following exemplary embodiments are presented:
1. A method of preparing a homogenous dispersion of aqueous droplets of a radiopharmaceutical or radiodiagnostic agent within a non-aqueous medium comprising the steps of:
   (a) dispersing dry Sodium Thiosulfate and dry Potassium Phosphate Dibasic Anhydrous within non-aqueous, liquid polyethylene glycol,
   (b) adding the radiopharmaceutical or radiodiagnostic agent within an aqueous liquid media to dissolve said dry Sodium Thiosulfate and dry Potassium Phosphate Dibasic Anhydrous within the non-aqueous, liquid polyethylene glycol, and to form a mixture of non-aqueous polyethylene glycol and an aqueous solution,
   (c) exposing said mixture to sonification and/or homogenization until a homogenous dispersion of aqueous droplets is formed.
      wherein said aqueous droplets comprise a solution of dissolved Sodium Thiosulfate, Potassium Phosphate Dibasic Anhydrous and radiopharmaceutical.
2. The method of embodiment 1 wherein said mixture is mechanically stirred.
3. The method of embodiment 1 wherein said radiopharmaceutical comprises 1-131.
4. The method of embodiment 1 wherein said radiodiagnostic agent comprises 1-131.
5. A method of preparing a homogenous dispersion of aqueous droplets of a radiopharmaceutical within a non-aqueous medium comprising the steps of:
   (a) dispersing dry salts within non-aqueous, liquid polyethylene glycol,
   (b) adding the radiopharmaceutical or radiodiagnostic agent within an aqueous liquid media to dissolve said dry salts within non-aqueous, liquid polyethylene glycol, and to form a mixture of non-aqueous polyethylene glycol and an aqueous solution,
   (c) exposing said mixture to sonification or homogenization until a homogenous dispersion of aqueous droplets is formed.
6. The method of embodiment 5 wherein said dry salts comprise Sodium Thiosulfate and Potassium Phosphate Dibasic Anhydrous.
7. The method of embodiment 5 wherein said mixture is mechanically stirred.
8. The method of embodiment 5 wherein said radiopharmaceutical comprises 1-131.
9. The method of embodiment 5 wherein said radiodiagnostic agent comprises 1-131.
10. The method of embodiment 1 wherein dispersing of dry Sodium Thiosulfate and dry Potassium Phosphate Dibasic Anhydrous within liquid polyethylene glycol (PEG) is accomplished by
   (a) placing a first layer of PEG on the bottom of a mixing vessel,
   (b) placing a second layer comprising PEG, dry Sodium Thiosulfate and dry, Potassium Phosphate Dibasic Anhydrous on top of said first layer,
   (c) placing a third layer of PEG on top of said second layer,
   (d) melting said PEG by applying heat,
   (e) stirring the contents of said mixing vessel until said dry Sodium Thiosulfate and dry Potassium Phosphate Dibasic Anhydrous are dispersed throughout said molten PEG.
11. The method of embodiment 5 wherein dispersing of dry salts within liquid polyethylene glycol (PEG) is accomplished by
   (a) placing a first layer of PEG on the bottom of a mixing vessel,
   (b) placing a second layer comprising PEG and dry salts on top of said first layer,
   (c) placing a third layer of PEG on top of said second layer,
   (d) melting said PEG by applying heat,
   (e) stirring said layers within said mixing vessel until said dry salts are dispersed throughout said molten PEG.
12. The method of any of the preceding embodiments wherein the particle size of one or more of said dry salts is controlled.
13. The method of embodiment 12 wherein the particle size is controlled by grinding and sieving one or more of said dry salts.
14. The method of embodiment 12 wherein one or more of said dry salts comprise particles of less than 590 μm.
15. The method of any of the preceding embodiments wherein said second layer comprising PEG and dry salts is mixed separately and then added to said mixing vessel.
16. The method of any of the preceding embodiments wherein said radiopharmaceutical or radiodiagnostic agent is any water soluble radiopharmaceutical or radiodiagnostic agent.
17. The method of any of the preceding embodiments wherein the aqueous liquid media provides a high pH.
18. The method of embodiment 17, wherein the aqueous liquid media comprises NaOH.
19. A composition comprising a homogenous dispersion of aqueous droplets of radiopharmaceutical or radiodiagnostic agent and suitable excipients which is stable for at least 20 minutes.
20. A composition comprising a homogenous dispersion of aqueous droplets of radiopharmaceutical or radiodiagnostic agent and suitable excipients which is stable for at least 1.5 hours.
21. A composition comprising a homogenous dispersion of aqueous droplets of radiopharmaceutical or radiodiagnostic agent and suitable excipients which is stable for a time sufficient to fill and solidify within gel capsules.
22. The composition or method of any of the preceding embodiments wherein said homogenous dispersion of aqueous droplets has a substantially uniform distribution of sodium thiosulfate.
23. The composition or method of any of the preceding embodiments wherein said homogenous dispersion of aqueous droplets has a substantially uniform distribution of radiopharmaceutical.

1. A method of preparing a homogenous dispersion of aqueous droplets of a radiopharmaceutical or radiodiagnostic agent within a non-aqueous medium comprising the steps of:
(a) dispersing dry Sodium Thiosulfate and dry Potassium Phosphate Dibasic Anhydrous within non-aqueous, liquid polyethylene glycol,
(b) adding the radiopharmaceutical or radiodiagnostic agent within an aqueous liquid media to dissolve said dry Sodium Thiosulfate and dry Potassium Phosphate Dibasic Anhydrous within the non-aqueous, liquid polyethylene glycol, and to form a mixture of non-aqueous polyethylene glycol and an aqueous solution,
(c) exposing said mixture to sonification and/or homogenization until a homogenous dispersion of aqueous droplets is formed.
wherein said aqueous droplets comprise a solution of dissolved Sodium Thiosulfate, Potassium Phosphate Dibasic Anhydrous and radiopharmaceutical.

2. The method of claim 1 wherein said mixture is mechanically stirred.

3. The method of claim 1 wherein said radiopharmaceutical comprises I-131.

4. The method of claim 1 wherein said radiodiagnostic agent comprises I-131.

5. A method of preparing a homogenous dispersion of aqueous droplets of a radiopharmaceutical within a non-aqueous medium comprising the steps of:
(a) dispersing dry salts within non-aqueous, liquid polyethylene glycol,
(b) adding the radiopharmaceutical or radiodiagnostic agent within an aqueous liquid media to dissolve said dry salts within non-aqueous, liquid polyethylene glycol, and to form a mixture of non-aqueous polyethylene glycol and an aqueous solution,
(c) exposing said mixture to sonification or homogenization until a homogenous dispersion of aqueous droplets is formed.

6. The method of claim 5 wherein said dry salts comprise Sodium Thiosulfate and Potassium Phosphate Dibasic Anhydrous.

7. The method of claim 5 wherein said mixture is mechanically stirred.

8. The method of claim 5 wherein said radiopharmaceutical comprises I-131.

9. The method of claim 5 wherein said radiodiagnostic agent comprises I-131.

10. The method of claim 1 wherein dispersing of dry Sodium Thiosulfate and dry Potassium Phosphate Dibasic Anhydrous within liquid polyethylene glycol (PEG) is accomplished by
(a) placing a first layer of PEG on the bottom of a mixing vessel,
(b) placing a second layer comprising PEG, dry Sodium Thiosulfate and dry, Potassium Phosphate Dibasic Anhydrous on top of said first layer,
(c) placing a third layer of PEG on top of said second layer,
(d) melting said PEG by applying heat,
(e) stirring the contents of said mixing vessel until said dry Sodium Thiosulfate and dry Potassium Phosphate Dibasic Anhydrous are dispersed throughout said molten PEG.

11. The method of claim 5 wherein dispersing of dry salts within liquid polyethylene glycol (PEG) is accomplished by
(a) placing a first layer of PEG on the bottom of a mixing vessel,
(b) placing a second layer comprising PEG and dry salts on top of said first layer,
(c) placing a third layer of PEG on top of said second layer,
(d) melting said PEG by applying heat,
(e) stirring said layers within said mixing vessel until said dry salts are dispersed throughout said molten PEG.

12. The method of any of the preceding claims wherein the particle size of one or more of said dry salts is controlled.

13. The method of claim 12 wherein the particle size is controlled by grinding and sieving one or more of said dry salts.

14. The method of claim 12 wherein one or more of said dry salts comprise particles of less than 590 μm.

15. The method of any of the preceding claims wherein said second layer comprising PEG and dry salts is mixed separately and then added to said mixing vessel.

16. The method of any of the preceding claims wherein said radiopharmaceutical or radiodiagnostic agent is any water soluble radiopharmaceutical or radiodiagnostic agent.

17. The method of any of the preceding claims wherein the aqueous liquid media provides a high pH.

18. The method of claim 17, wherein the aqueous liquid media comprises NaOH.

19. A composition comprising a homogenous dispersion of aqueous droplets of radiopharmaceutical or radiodiagnostic agent and suitable excipients which is stable for at least 20 minutes.

20. A composition comprising a homogenous dispersion of aqueous droplets of radiopharmaceutical or radiodiagnostic agent and suitable excipients which is stable for at least 1.5 hours.

21. A composition comprising a homogenous dispersion of aqueous droplets of radiopharmaceutical or radiodiagnostic agent and suitable excipients which is stable for a time sufficient to fill and solidify within gel capsules.

22. The composition or method of any of the preceding claims wherein said homogenous dispersion of aqueous droplets has a substantially uniform distribution of sodium thiosulfate.

23. The composition or method of any of the preceding claims wherein said homogenous dispersion of aqueous droplets has a substantially uniform distribution of radiopharmaceutical.
